(19) United States
(12) Patent Application Publication
(10) Pub. No.: US 2010/0286708 A1
(43) Pub. Date: Nov. 11, 2010

(54) System for blood vessels cleaning, such as for a coronary artery, peripheral artery or any other body vessel, based on mobile agent

(76) Inventor: Dan Rittman, Atlit (IL)

Correspondence Address: DANNY RITTMAN, Amat Ha Maim 23B, Atlit, 30300, IL

(21) Appl. No.: 12/387,593

(22) Filed: May 6, 2009

(51) Int. Cl. A61B 17/22

(52) U.S. Cl. 606/127; 606/159

(57) ABSTRACT

This paper describes a system and method invention for blood vessels cleaning, such as a coronary artery, peripheral artery, or any other body vascular based on a mobile agent. The system consists of device which is electronically controlled, performing a procedure of vessel walls cleaning by removing the accumulated plaque or any other blockage material. This plaque or other blockage type may cause atherosclerosis and can eventually cause the artery to be blocked, causing heart disease, heart attack or stroke. Upon initial setup the system will be inserted into a human body and travel within its blood vessels. Based on sensors the system finds clogged areas and remove the blockage, without damaging the vessel's tissue using its cleaning system. The system independently and/or programmatically travels and searches for blockages within blood vessels using its own propulsion and navigation system. The system includes also a mechanism to remove and collect the removed plaque or any other blockage material. This system performs an efficient blood vessels cleaning and provides an innovative non surgical method for blood vessels blockage removal.

### TECHNICAL FIELD OF THE INVENTION

The present invention relates generally to the field of vascular therapies. More particularly, the invention relates to strategies for treating a plaque or any other blockage material associated with a blood vessel of a patient.

### FIELD AND BACKGROUND OF THE INVENTION

Heart disease, specifically coronary artery disease (CAD), is a major cause of death, disability, and healthcare expense. Until recently, most heart disease was considered to be primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to heart muscles, causing a heart attack. Alternatively, the clot may break off and lodge in another organ vessel such as the brain resulting in a thrombotic stroke.

Within the past decade, evidence has emerged expanding the paradigm of atherosclerosis, coronary artery disease, and heart attacks. While the buildup of hard plaque may produce angina and severe ischemia in the coronary arteries, new clinical data now suggests that the rupture of sometimes non-occlusive, vulnerable plaques causes the vast majority of heart attacks. The rate is estimated as high as 60-80 percent. In many instances vulnerable plaques do not impinge on the vessel lumen, rather, much like an abscess they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

Atherosclerotic plaques vulnerable to rupture are typically small deposits covered by thin fibrous caps (less than 70 microns) covering lipid cores. Within the fibrous cap is a dense infiltrate of smooth muscle cells, macrophages and lymphocytes. The lipid pool is believed to be formed as a result of a pathological process involving low density lipoprotein (LDL), macrophages, and the inflammatory process. The macrophages oxidize the LDL producing foam cells. The macrophages, foam cells, and smooth muscle cells sit beneath the endothelium and release various toxic substances, such as tumor necrosis factor and tissue factor. These substances damage the arterial wall and surrounding areas and can result in generalized cell necrosis and apoptosis, pro-coagulation, and weakening of the fibrous cap. The inflammation process may weaken the fibrous cap to the extent that sufficient mechanical stress, such as that produced by increased blood pressure, may result in rupture. The lipid core and other contents of the vulnerable plaque (emboli) may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

Several strategies have been developed for the detection (e.g., diagnosis and localization) of vulnerable plaques. One strategy involves the measurement of temperature within a blood vessel. For example, vulnerable plaque tissue temperature is generally elevated compared to healthy vascular tissue. Measurement of this temperature discrepancy may allow detection of the vulnerable plaque.

Another detection strategy involves labeling vulnerable plaque with a marker. The marker substance may be specific for a component and/or characteristic of the vulnerable plaque. For example, the marker may have an affinity for the vulnerable plaque, more so than for healthy tissue. Detection of the marker may thus allow detection of the vulnerable plaque. Alternatively, the marker may not necessarily have an affinity for the vulnerable plaque, but will simply change properties while associated with the vulnerable plaque. The property change may be detected and thus allow detection of the vulnerable plaque.

Regardless of the strategy used for detection, a formidable problem remains in the treatment of the vulnerable plaque. Without appropriate treatment, the vulnerable plaque may rupture and subsequently release embolic material and cause great risk to the patient. Drug and other therapies exist that may reduce the size and chance of vulnerable plaque rupture over a relatively long time frame. These therapies, however, may not be desirable or effective for all patients, including those having vulnerable plaques on the immediate verge of rupture. With such therapies, accidental or unanticipated rupture of these truly vulnerable plaques may occur in a non-clinical setting.

Therefore, it would be desirable to provide a system that would enable the removal of vulnerable and hard plaque. Furthermore, it would be desirable for such a system to periodically check for any potential embolic material from escaping a ruptured vulnerable plaque and causing risk to the patient.

### SUMMARY OF THE INVENTION

A first aspect according to the invention provides a system and method of removing a hard and vulnerable plaque associated with a blood vessel of a patient. The method includes inserting a wire bases or autonomous, robotic device within a lumen of the blood vessel. The autonomous device is automatically or remotely controlled searches for hard or vulnerable plaque or any other blockage material adjacent a wall of the blood vessel. Once a blockage is found, the system calculates its size and risk factor and decides about its removal.

The system autonomously or remotely controlled, navigates within blood vessels to search for blood clots using its own, electronically controlled, propulsion and navigation system.

A second aspect according to the invention provides a system for removing a hard and vulnerable plaque associated with a blood vessel of a patient. The system includes a removal system which is based on computerized, electronic system to analyze the blockage size and its risk factor and prepare a matched removal plan. The removal system consists of laser or ultrasonic or thermal or mechanical sub-systems to perform the blockage removal without causing any damage to the blood vessel wall.

A third aspect according of the invention provides a sensors system to calculate the blockage size, risk factor and the removal plan. Another sensor system supervises the removal plan to ensure safe operation without damaging the blood vessel wall. Another sensor system controls the removed blockage material collection in order to avoid floating debris in the blood stream.

A fourth aspect according to the invention provides a system for removing a hard and vulnerable plaque associated with a blood vessel of a patient. The system includes sensor system for independent navigation of the autonomous, robotic system within blood vessel. Another sensor system finds blockage and positioning the device within a lumen of the blood vessel and operating the removal system of the device without damaging the wall of the blood vessel.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 schematically illustrates a 2D view of a coronary artery from a coronary CT angiogram. The coronary artery shown contains hard or calcified plaque associated with a blood vessel of a patient, in accordance with the present invention;

FIG. 2 schematically illustrates a 2D view of a coronary artery from a coronary CT angiogram. The coronary artery shown contains soft or uncalcified plaque which is vulnerable to rapture, associated with a blood vessel of a patient, in accordance with the present invention;

FIG. 3 schematically illustrates the system for hard and vulnerable plaque or any other blockage material, detection sub-system within a patient, in accordance with the present invention;

FIG. 4 schematically illustrates the system for hard and vulnerable plaque or any other blockage material, removal system within a patient, in accordance with the present invention;

FIG. 5 schematically illustrates the system for debris collection of hard and vulnerable plaque or any other blockage removed material, within a patient, in accordance with the present invention;

### DESCRIPTION OF THE PREFERRED EMBODIMENT

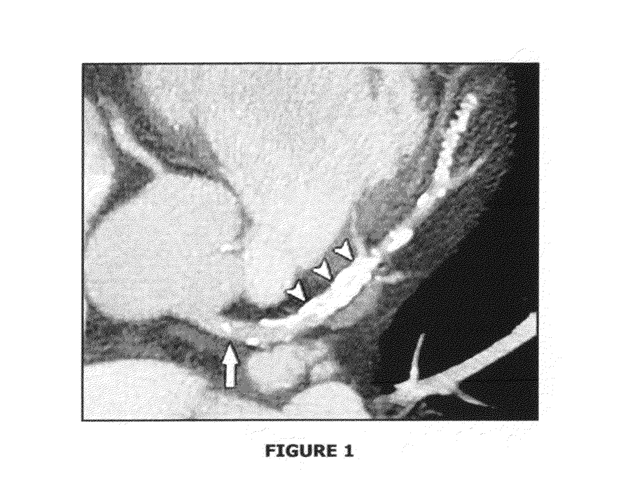

Referring to FIG. 1, conceptually illustrates is a 2D view of a coronary artery from a coronary CT angiogram. The coronary artery shown contains hard or calcified plaque. The hard plaque appears bright white and is marked with arrow heads. Hard plaque is more stable than soft plaque.

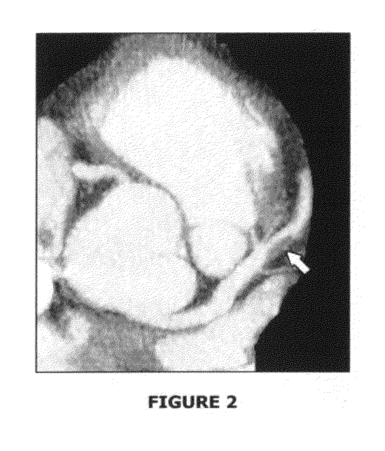

Referring to FIG. 2, conceptually illustrates is a 2D view of a coronary artery from a coronary CT angiogram. The coronary artery shown contains soft or uncalcified plaque. The soft plaque appears gray and is marked with an arrow. Soft plaque is more vulnerable to rupture and cause a heart attack than hard plaque.

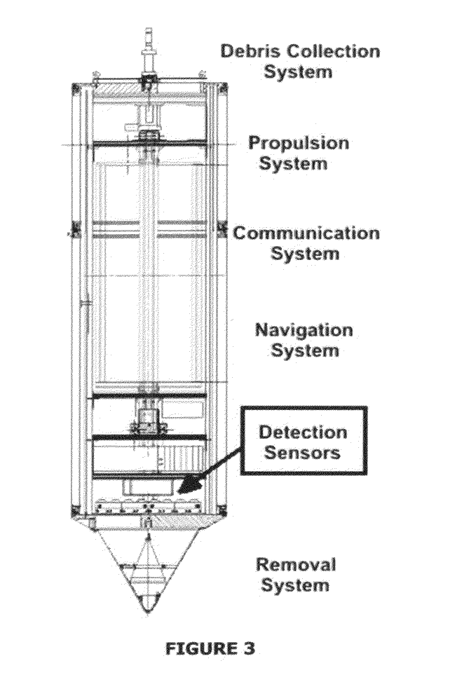

Referring to FIG. 3, conceptually illustrates the system for hard and vulnerable plaque or any other blockage material, detection sub-system within a patient, in accordance with the present invention; The system includes electronically controlled sensors sub-system to detect blood vessels blockage which may cause a risk of heart attack, stroke or any other fatal phenomenon to the patient.

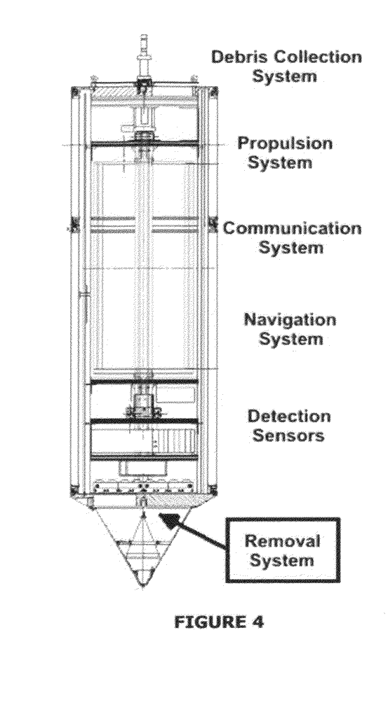

Referring to FIG. 4, conceptually illustrates the system for hard and vulnerable plaque or any other blockage material, removal sub-system within a patient, in accordance with the present invention; the system includes electronically controlled sub-system to remove blood vessels blockage, using laser, ultrasonic, thermal or mechanical method.

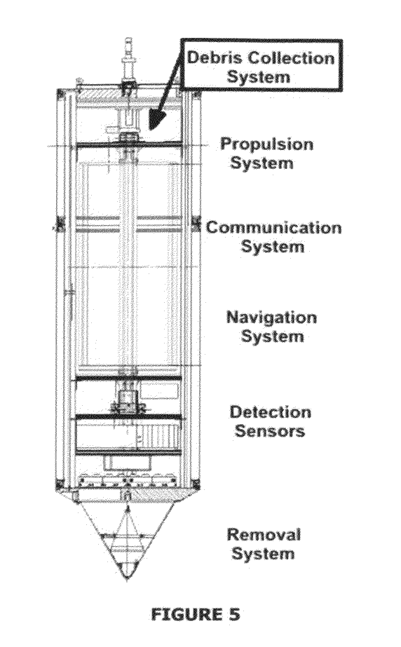

Referring to FIG. 5, conceptually illustrates the system for hard and vulnerable plaque or any other blockage material, debris collection sub-system within a patient, in accordance with the present invention; the system includes electronically controlled sub-system for debris collection of the removed blockage's material within blood vessels.

1. A system comprising: A device that performs cleaning of any type of blood vessels blockages, said method comprising:
A device that is inserted wireless or using flexible wire into blood vessels; and,
A device which includes electronically controlled based system to travel within blood vessels; and,
Electronically controlled sensors system to navigate within the blood vessels; and,
Electronically controlled sensors system to calculate constantly the blood vessels thickness; and,
Electronically controlled sensors system to search for vessels blockages; and,
Electronically controlled sensors system to determine a blockage within the blood vessels.

2. In case of blockage identification, the system calculates the blockage's dimensions and prepares a removal plan, taking into account the artery's tissue thickness to avoid tissue damage.

3. The system of claim 1 includes clogging material removal mechanism which is laser, ultrasonic, thermal, mechanical based or other method to smoothly remove the blood vessel blockage, typically defined as plaque or any other blockage material.

4. The system in claim 1 includes autonomous, mobile robotic device.

5. The system in claim 1 includes wire inserted based device.

6. The system in claim 1 includes electronically controlled, remote operation, mobile device.

7. The system in claim 1 includes blockage debris collection system.

8. The system in claim 1 includes electronic controlled propulsion system to autonomously search, find and clean all the vessel's blockages.

9. The system of claim 1 includes intelligent mobile software to communicate, interact and collaborate with external electronic system creating an optional remote system to control the robotic device.

10. An embedded computer program comprising: A computer program on flash memory medium having a computer readable program code for navigating the device within blood vessels, wherein the computer program controls micro-motors and mechanical sub-systems to maneuver the robotic device within the blood vessel.

11. A computer program code to control ultra-sonic sensors system to continuously calculate the blood vessel tissue thickness and;
computer program for calculating the blockage size and;
computer program to prepare and execute a blockage cleaning plan and;
computer program to control the scrubbing mechanism for smooth removal of the blockage.
computer program to control the blockage debris collection.

12. The system of claim 1 includes mini-camera to transmit the inner blood vessel view into an outside display system, comprising:
(a) The outside display is Liquid Crystal Display monitor.
(b) The outside system includes an electronic system to convert the information transmitted by the robotic camera into a real-time image of the inner blood vessel.
(d) The robotic mini-camera included controlled zoom mechanism that can be controlled by the outside electronic system via wireless system.

13. The robotic on-board computer program that is controlling an on-board electronic system and provides vital medical information to the outside system via wireless, comprising:
(a) Blood type information.
(b) Blood temperature information.
(c) Vessel tissue thickness information.
(d) Blockage size and type information.
(e) Suggestions for blockage removal.
(f) Continuous status report during blockage removal procedure.

14. The autonomous robotic system includes emergency shut-down system, comprising:
(a) Electronic system to identify risky situations during blockage removal and stop the cleaning procedure.
(b) Electronic system to identify accidental blood vessel tissue damage during the cleaning and stop the cleaning procedure.
(c) Electronic system that receives an Emergency Shut-Down command from the outside, remote system to stop the procedure.

15. The autonomous system of claim 1 is shaped as a bullet to avoid inner blood vessel damage.

16. The autonomous system of claim 1 is operated on internal battery based power source.

17. The system includes a mechanism for taking a sample of the blockage specimen for further analysis that may be done when the robotic system exits the blood vessel, comprising:
(a) Mechanical system that is electronically controlled to collect blockage specimen.
(b) Computer program to control the mechanical system.

18. The system of claim 1 including illumination system to light the inner blood vessel, comprising:
(a) Micro-LED illumination source to illuminate the inner blood vessel.
(b) Computer program to control the light intensity of the Micro-LED illumination source.
